(19) United States
(12) Patent Application Publication
(10) Pub. No.: US 2008/0193917 A1
(43) Pub. Date: Aug. 14, 2008

(54) Biological Detection System and Method

(76) Inventors: David Alexander Schofield, Hollywood, SC (US); Augustine Anthony Dinovo, James Island, SC (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/914,431

(22) PCT Filed: May 26, 2006
(86) PCT No.: PCT/US06/20382

§ 371 Date: Nov. 14, 2007

Related U.S. Application Data
(60) Provisional application No. 60/685,112, filed on May 27, 2005.

(51) Int. Cl. C12Q 1/70; C12M 1/00

(52) U.S. Cl. 435/5; 435/287.1; 435/287.2

(57) ABSTRACT

The present disclosure provides a biological detection system and method. One detection system may include a genetically engineered phage that expresses a surface molecule able to bind a target molecule; a bacterium susceptible to infection by the phage; and a detection component able to determine whether the bacterium has been infected by the phage. Infection of a bacterium by a phage may be indicative of phage binding to the target molecule. One method may include placing a sample suspected of containing the target molecule with a binder; adding a genetically engineered phage having reporter genetic material and able to bind the target molecule; washing away unbound phage; releasing phage bound to the target molecule; infecting a bacterium with the released phage; and detecting the presence of any reporter genetic material in the bacterium. Reporter material in the bacterium may correlate with target molecule in the sample.

### CROSS-REFERENCE TO RELATED APPLICATIONS

This application claims the benefit of U.S. Provisional Application No. 60/685,112 filed on May 27, 2005, entitled “Biological Detection System and Method”, which is incorporated herein by reference in its entirety.

### TECHNICAL FIELD

The present disclosure, according to one embodiment, relates to a biological detection system and method. In a more specific embodiment it relates to a detection system and method using phage binding and bacterial infection to detect the presence of a target molecule.

### BACKGROUND

Toxins may be identified using mouse bioassays, in which toxin specific antibodies protect the mice against the lethal action of the toxin. These bioassays, although very sensitive, may take 1-4 days to complete and require animal testing. Other types of toxin (e.g., seafood toxins) detection methods may require expensive laboratory equipment (mass spectrometry) and expertise for use and may not be suitable for use in nonlaboratory environments.

Newer detection and identification methodologies include polymerase chain reaction (PCR) assays and immunoassays. PCR assays are very specific and sensitive due to the amplification process and theoretically, may detect as little as one target molecule; however, PCR assays generally require some target preparation, and are not useful for the detection of purified toxins since they lack the necessary genetic information.

### SUMMARY

Current biological detection methods are often expensive, time consuming and require expensive laboratory equipment and expertise. In addition, these detection methods are often limited in their sensitivity and require large amounts of the target material. New rapid and sensitive detection methodologies may contribute to saving lives and/or reducing costs. Embodiments of the present disclosure provide a biological detection system and method.

In one embodiment, the disclosure provides a detection system. The system may include a phage (e.g., a genetically engineered phage). The phage may express a surface molecule having a binding affinity for a target molecule (e.g., K_{d}≧10⁻⁵ M, K_{d}≧10⁻⁶ M, K_{d}≧10⁻⁷ M, K_{d}≧10⁻⁸ M, or K_{d}≧10⁻⁹ M). The system may also include a binder with a binding affinity for a target molecule (e.g., K_{d}≧10⁻⁵ M, K_{d}≧10⁻⁶ M, K_{d}≧10⁻⁷ M, K_{d}≧10⁻⁸ M, or K_{d}≧10⁻⁹ M). The system may further include a composition formulated to permit binding of a target molecule to a phage and/or binder. These compositions may be additionally formulated to solubilize phage that is not bound to a target molecule and/or a binder. Thus, such compositions may be used to wash a binder and/or target molecule to remove unbound phage. In some embodiments, a composition may be a phage wash composition formulated to permit binding of a phage surface molecule to a target molecule-binder complex, but solubilize, suspend, or otherwise separate surplus phage from the binder. In other embodiments, a composition may be a release composition formulated to solubilize, suspend, or otherwise dissociate a phage from a binder. A target-molecule release composition may optionally be formulated to permit phage surface molecule—target molecule binding. A system may additionally include a cell (e.g., a bacterium) capable of taking up at least a portion of a phage (e.g., a phage nucleic acid). In some embodiments, a cell may be susceptible to infection by a phage. A system may further include a detector configured to differentiate between cells that have taken up at least a portion of a phage and those that have not (e.g., phage-infected cells versus non-infected cells). Infection of a cell by a phage may be indicative of phage binding to the target molecule.

In another embodiment, the disclosure provides a method of detecting the presence of a target molecule. The method may include:
(a) contacting a binder with a sample possibly comprising a target molecule to form a binder—sample mixture under conditions that permit binder—target molecule binding;
(b) contacting the binder—sample mixture with a phage (e.g., a genetically engineered phage) having reporter genetic material, wherein the phage is capable of binding the target molecule; under conditions that permit phage—target molecule binding to form a binder—sample—phage mixture,
(c) washing the binder—sample—phage mixture under conditions that remove phage not bound to the target molecule from the binder (e.g., using a phage wash composition) to form a washed binder—sample—phage mixture;
(d) treating the washed binder—sample—phage mixture to release phage bound to the target molecule, if any, from the binder (e.g., by contacting the phage with a release composition) to form a released phage composition;
(e) contacting a bacterium with the released phage composition under conditions that permit uptake of at least a portion of the released phage, if present, by the bacterium (e.g., infection), and
(f) detecting the presence of any reporter genetic material in the bacterium.
   The presence of reporter genetic material in the bacterium may be indicative of presence of a target molecule in the sample, while absence of reporter genetic material in the bacterium may be indicative of absence of the target molecule in the sample.

In some embodiments, a released phage composition may include one or more materials (e.g., detergents, solvents, salts) that may disfavor phage uptake by a bacterium and/or interfere with bacterial health and/or replication (collectively “interferant”). Accordingly, a phage release composition may be treated to reduce or remove at least a portion of the interferant(s) present, if any. Conditions that permit uptake of at least a portion of the released phage may include, according to some embodiments, pretreating or conditioning a released phage composition to have a desired composition, pH, ionic strength, temperature and/or other property to form a treated phage release composition.

In some embodiments, the detection system or method may be as sensitive as PCR. Without being limited to any particular mechanism of action, this sensitivity may be due, in whole or in part, to the possible amplification when phage expand in a single, infected bacterium and/or when a bacterium infected by a phage multiplies. In additional embodiments, a detection system and method may be easily and rapidly adapted to detect any toxin of interest simply by changing the binding molecule (e.g., peptide, antibody, nucleic acid, or fragment thereof) on the cell surface of the phage and/or one or more binder components.

Systems, methods, and/or devices, according to some embodiments of the disclosure, may be configured to permit rapid detection of a target molecule. For example, a target molecule may be detected in less than about twelve (12) hours, less than about ten (10) hours, less than about eight (8) hours, less than about six (6) hours, or less than about four hours. A target molecule may be detected in less than about three (3) hours, less than about two (2) hours, or less than about one (1) hour. The time required for detection may be a function of the time required for target binding, sample loading, washing, elution, infection, and/or reporter detection. For example, detection time may be reduced by electing to use columns, which permit reagents to flow through them, instead of plates, which may require more labor intensive manual application and removal of reagents.

Systems, methods, and/or devices, according to some embodiments of the disclosure, may be configured to permit sensitive detection of a target molecule. Sensitivity may be assessed in terms of the number of detectable eluted phage. For example, systems, methods, and/or devices of the disclosure may be configured to detect less than about five hundred (500) eluted phage, less than about one hundred (100) eluted phage, less than fifty (50) eluted phage, or less than twenty-five (25) eluted phage. In some specific examples, less than about ten (10) eluted phage or less than about five (5) eluted phage may be detected. In some embodiments, about 10⁻¹⁷ g of a target molecule (e.g., toxin) may be detected.

### BRIEF DESCRIPTION OF THE DRAWINGS

Some specific example embodiments of the disclosure may be understood by referring, in part, to the following description and the accompanying drawings, wherein:

FIG. 1 is a schematic of a toxin detection system and method, according to a specific example embodiment of the present disclosure.

FIG. 2 illustrates an embodiment of the disclosure in which the functionality of the plasmid luxAB cassette was assessed by examining Escherichia coli (E. coli) cells under dark field illumination to view luminescent colonies;

FIG. 3 illustrates a schematic map of a LuxABM13 plasmid in accordance with an embodiment of the disclosure;

FIG. 4 illustrates a comparison of the latency period between a luxABM13 and wild-type M13 in accordance with an embodiment of the disclosure;

FIG. 5 illustrates phage infection during various stages of E. coli growth in accordance with an embodiment of the disclosure;

FIG. 6 illustrates luxABM13 infection at various E. Coli densities in accordance with an embodiment of the disclosure;

FIG. 7 illustrates signal response time from phage addition to detectable light above background in accordance with an embodiment of the disclosure;

FIG. 8A illustrates a dose response curve with from 5×10² to 10⁵ phage in accordance with an embodiment of the disclosure;

FIG. 8B illustrates a dose response curve with from 5×10⁶ to 10⁹ phage in accordance with an embodiment of the disclosure;

FIG. 9 illustrates assay sensitivity in accordance with an embodiment of the disclosure;

FIG. 10 illustrates LuxABM13 latency using cells stored under various conditions in accordance with an embodiment of the disclosure;

FIG. 11 illustrates light production and signal response times using cells stored under various conditions in accordance with an embodiment of the disclosure; and

FIG. 12 illustrates luciferase activity at various temperatures in accordance with an embodiment of the disclosure.

While the present disclosure is susceptible to various modifications and alternative forms, specific example embodiments thereof have been shown in the drawings and are herein described in detail. It should be understood, however, that the description herein of specific example embodiments is not intended to limit the disclosure to the particular forms disclosed herein, but on the contrary, this disclosure is to cover all modifications and equivalents.

### DETAILED DESCRIPTION

The present disclosure, according to one embodiment, relates to a biological detection system and method using phage binding and bacterial infection.

One embodiment relates to a detection system including a phage and a bacteria that may be infected by the phage. The phage may be genetically engineered to express a particular surface molecule. This surface molecule may be capable of binding to a target molecule under certain conditions, then being released from the target molecule under other conditions without destroying the phage's ability to infect the bacteria. In some embodiments, the surface molecule may include a peptide. In other embodiments, a surface molecule may include an antibody or antibody fragment. In still other embodiments, surface molecule may include a carbohydrate, a nucleotide, and/or a lipid.

A system may also include a binder for the target molecule that binds the target molecule under conditions that also allow phage binding. The binder may be specific for the target molecule or non-specific. The binder may be unable to bind significant amounts of the phage without the target molecule. The system may have a washing component able to wash unbound phage from the binder and a release component which supplies the conditions for release of the phage from the binder. Additionally, the system may include a detection component able to determine if the bacteria have been infected by the phage and thus whether any target molecule was present on the binder. The detection component may be able to distinguish how many bacteria have been infected and, for this information, determine an amount of target molecule present on the binder. In some embodiments, the system may be so sensitive that the presence of a single target molecule or the infection of bacteria with a single phage may be detected. Thus, according to some embodiments, a system may exhibit the robust and easy to use nature of immunoassays but display the sensitivity of PCR.

The detection system may be provided as a kit. For example, the kit may contain at least a phage, a binder, and a bacteria. The kit may also be in the form of an automated portable light sensor device that may be used outside of a laboratory for the detection of a target molecule. Such a device may be particularly useful for detection of environmental toxins, for example in military situations, in suspected terrorist attacks, monitoring of local water and/or food supply, monitoring of disease outbreaks, or in suspected environmental or other disasters.

Phage may be resistant to environmental extremes and may be stored for months or years without a significant loss in phage infectivity. A bacteria, however, may loose its susceptibility to phage infection after storage for long periods of time. Some bacteria may regain their susceptibility to phage infection. Thus, the signal response time for bacteria may depend, in part, upon storage periods in kits and storage conditions.

The disclosure provides methods of detecting the presence of a target molecule. The sample suspected of containing a target molecule may be placed in conditions that allow binding of the target molecule to a binder. The binder may be specific to the target molecule or non-specific. The binder may be unable to bind appreciable amounts of phage in the absence of the target molecule. The binder may then be placed in the presence of a genetically engineered phage under conditions that allow continued binding of the target molecule to the binder, but that also allow binding of the phage to the target molecule, if present. The binder may then be washed under conditions sufficient to remove a substantial portion or all of any phage not bound to a target molecule without interrupting binding between the binder and target molecule and target molecule and phage. Then, the phage may be released by washing the binder under conditions that allow release of the phage from the target molecule. Conditions may be selected such that, after release, the phage remains able to infect a bacterium with reporter genetic material. The released phage may be used to infect a bacterium or bacteria with the reporter genetic material. The presence of this reporter generic material may then be detected. For example, the reporter genetic material may confer luminescence upon the infected bacteria, which may then be detected. Reporter genetic material may also be further amplified after infection, for example by expansion of the phage in the infected bacteria followed by further infection of nearby bacteria.

The number of eluted phage, in some embodiments, may be the same or substantially the same as the number of phage to which a target molecule was bound (e.g., the number of binder—target molecule—phage complexes applied to or on a column). One or more target molecules may bind to a single phage particle (e.g., where the surface of a phage includes more than one surface molecule and/or where each surface molecule binds two or more target molecules.

In a specific embodiment, M13 filamentous phage may be genetically engineered to create a reporter phage that also displays a specific binding peptide on its cell surface. The reporter phage may, for example, include genetic material able to cause infected bacteria to become luminescent. The genetically engineered phage may serve as a bioamplifiable tag that specifically binds the target molecule and amplifies the reporter signal (e.g. light) upon infection of E. coli. The infected E. coli may produce light and also amplify the signal by releasing 500-1000 new progeny phages which may infect other E. coli cells and produce more light.

Staphylococcal enterotoxin B (SEB), along with botulinum, ricin and ε-toxin may be among the most likely agents used as an aerosolized biological warfare agent against U.S. forces (Greenfield R A et al., (2002) Am J Med Sci 323, 326-340). SEB is one of five closely related enterotoxins (SEA, SEC, SED and SEE) produced and excreted by the human pathogen Staphylococcus aureus during growth. Although SEB may be inherently less toxic than, for example, botulinum toxin, patients exposed to SEB become seriously ill experiencing fever, shortness of breath, chest pain, vomiting and diarrhea. These conditions may incapacitate a fighting force for up to about two weeks. Higher toxin exposure may lead to septic shock and death. As a threat agent, SEB may be used primarily as an aerosol hazard; however, SEB may be a common causative agent of food poisoning upon digestion of improperly handled food. Therefore, SEB potentially may be delivered via inhalation, such as through aerosols, or ingestion, such as through deliberately contaminated water or food supplies. Both portals of toxin entry are toxic to humans and there is currently no human vaccine against SEB poisoning.

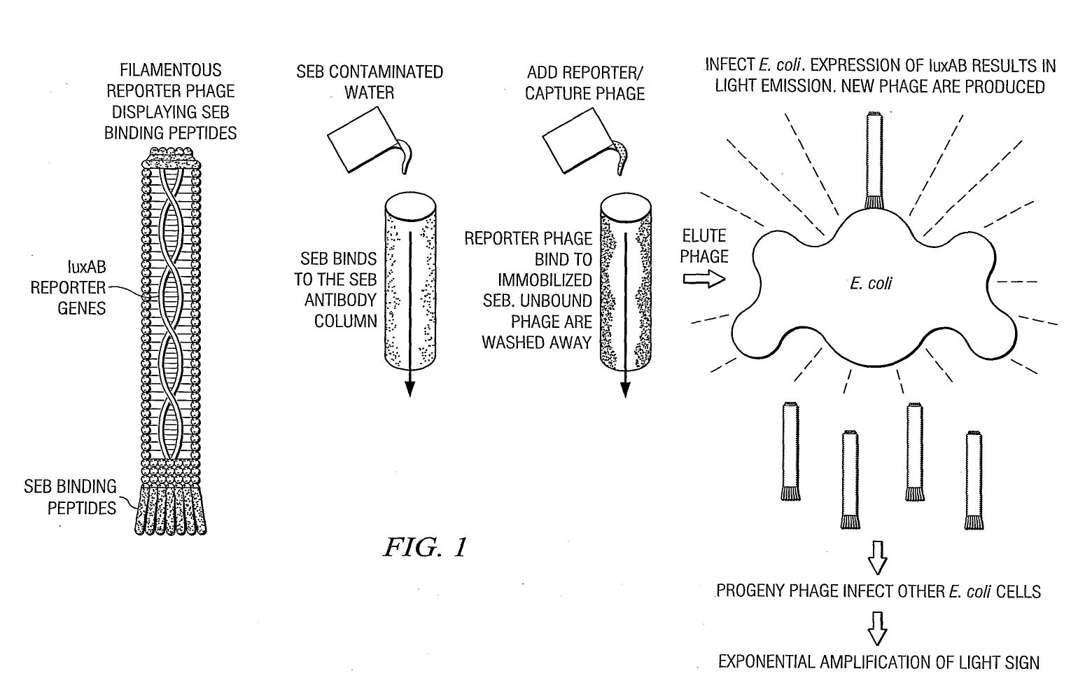

One specific example embodiment of the disclosure invention relates to the detection system of FIG. 1. In this example, the target molecule may include staphylococcal enterotoxin B (SEB) and the phage may contain reporter genes which may be the luxAB genes from Vibrio harveyi (e.g., Accession No. E12410). These genes may confer luminescent properties upon bacteria. The filamentous M13 phage may be used as a detector and bioamplifiable reporter for the detection of SEB. The M13 phage may be genetically engineered to contain the Vibrio harveyi luxAB genes to create a genetically engineered phage capable of causing infected bacteria to produce light. In addition, the genetically engineered phage may be modified to display a specific toxin binding peptide on its exterior surface or coat. Specifically, the peptide may bind SEB.

SEB contaminated samples or samples suspected of containing SEB may be liquefied, if needed, and passed through a column containing immobilized SEB specific antibodies. Only SEB may specifically bind to the antibodies while non-specific particles may pass through the column. The genetically engineered phage may then be passed through the column where it may specifically bind to any SEB present. Unbound phage may be washed away. The SEB-binding phage may be eluted from the column into a solution containing M13 phage competent E. coli cells. Phage infection of E. coli may result in phage DNA replication, luxAB reporter gene expression, light emission, the production of approximately 1000 new virions on the first infection cycle, or any combinations of the above. Newly produced virions may be extruded from the cell without cell lysis and may infect other E. coli cells, resulting in exponential amplification of the signal until a certain amount of time has passed or the number of E. coli hosts are exhausted.

Light emission may occur without processing of the sample and may be sensitively detected through multiple commercially available means without the use of expensive equipment or specific expertise. The amount of light produced and the time taken to generate the signal may be directly proportional to the amount of toxin present in the original sample. If no SEB is present in the sample, then no light may be detected. Alternatively, if the amount of SEB in the sample is below a certain threshold, then no light may be detected, particularly within a specific time frame.

In some embodiments, a binder may include an SEB binding peptide. For example, an SEB peptide may be isolated from a phage display library and exhibit strong binding to SEB (e.g., Goldman E R (2000) J Mol Recognit 13, 382-387). Specificity experiments using other staphylococcal enterotoxins (SEA, SEC and SED) with the peptide of Goldman, for example, showed no cross reactivity with SEA and SED but significant cross-reactivity with SEC, which shares approximately 65% sequence identity with SEB. Therefore, this capture phage may also detect SEC in addition to SEB. The DNA sequence encoding the SEB peptide (WHKAPRAPAPLL; SEQ ID NO. 1) may be cloned into the M13KE phage display vector (New England Biolabs, Inc., Beverly, Mass.) so that it will be fused in frame with the pIII coat protein sequence. The resulting phage may display five copies of the fusion peptide coat protein on the surface of each virion. Error prone PCR and phage display studies may be performed using the conserved SEB codons and randomizing the flanking residues in order to optimize the affinity and specificity of the peptide. The phage may also be genetically engineered to contain the V. harveyi luxAB reporter genes under the control of optimized E. coli transcriptional and translational expression signals to maximize reporter gene expression and light emission (Schofield D A et al., (2003) Appl Environ Microbiol 69, 3385-3392). The filamentous M13 phage may be an appropriate phage for this embodiment because: (i) Reporter genes such as luxAB may be inserted into the intergenic region of the M13 phage genome without any loss of phage function; (ii) M13 infects E. coli within 2 minutes and releases progeny phage after 20 minutes, thus offering a fast signal response time; (iii) M13 phage, in comparison to lytic bacteriophage, do not lyse their host, but instead extrude progeny virions through the bacterial membrane. Consequently, infected E. Coli hosts may continually produce light as well as amplify the signal by producing new progeny phage; (iv) M13 phage display vectors enabling cloning and in frame fusion with the phages coat protein are commercially available and (v) Amplification of the number of copies of the luxAB genes may occur immediately after phage infection because the single stranded phage DNA encoding the luxAB genes may be converted into multiple double stranded replicative forms.

A binder may include or be linked to, according to some embodiments a gel, bead, column, well, and/or other matrix. In some embodiments, a binder may be configured and arranged to permit rapid and/or automated separation of phage particles. For example, one or more columns may be configured to permit spinning along their longitudinal axis.

In another embodiment, phagemid vectors may be used instead of phage to encode the displayed molecule and the reporter genetic material. The phagemid vectors (which may contain a phage and plasmid origin of replication) may be grown as plasmids containing the genetic information for the reporter gene and displayed surface molecule and may be packaged into recombinant M13 phage with the aid of a helper phage such as M13KO7 or VCSM13. The recombinant phage may be used as described herein to infect bacteria and produce a signal if the target molecule was present. However, the recombinant phage may not be able to reinfect other bacteria and produce progeny phage. Under these conditions, helper phage such as M13KO7 and VCSM13 may be added to the infected bacteria in order to produce recombinant progeny phage and further amplify the signal.

In another embodiment, lytic phage may be used instead of filamentous phage to display the capture molecule on its surface. Alternatively, phage infection may be detected by the release of phage encoded proteins/recombinant proteins into the medium. The presence of the target molecule may be detected upon lytic phage infection of the target bacteria as indicated by rapid clearing (lysis) of the infected bacteria. Alternatively, phage infection may be detected by the release of bacterial components into the surrounding media. For example, bacterial lysis may be detected by an increase in the concentration of ATP in the medium released from the bacterial cell upon cell lysis.

luxAB may be selected as a reporter because expression in E. coli may be sensitively detected and/or because detection may require little to no processing of the bacteria. Detection may require addition of a substrate, such as the aldehyde substrate decanal, which may be readily capable of passing through the bacterial membrane and entering the cell. In addition, light detector instruments are readily available and may be used in this embodiment. A temperature controlled portable light sensor may also be used.

In another embodiment, a bacterium may include a reporter nucleic acid. A reporter nucleic acid in the bacteria may be operably linked to an expression control sequence (e.g., a promoter). An expression control sequence may be selected or configured to be active only in the presence of an activator protein, which the bacteria do not possess. A phage may be selected or configured to include an activator protein that is strictly required for expression of a reporter nucleic acid. In this embodiment, phage infection of bacteria may supply the activator protein, resulting in reporter gene expression which may be indicative of the target molecule being present.

In another embodiment, an expression control sequence operably linked to a reporter nucleic acid may include a repressible promoter (e.g., including repressor protein binding sites). The phage may be selected or configured (e.g., genetically modified) to include multiple sequences of a repressor protein binding site. In the absence of phage infection, the bacterial promoter may be inactive (repressed) such that there is no expression of the reporter nucleic acid. Upon phage infection, the phage DNA containing the multiple repressor binding site is present and amplified, which titrates the bacterial repressor protein leading to promoter activation and expression of the reporter nucleic acid.

In another embodiment, neither the phage or the bacteria may contain the reporter genetic material. Instead, the target signal may be detected after phage infection by the increase in progeny phage released by the bacteria. This may be achieved using anti M13 antibodies and a generic immunoassay/or a quantitative PCR assay using the phage DNA as template.

Both specific and nonspecific SEB column binding may be used. Use of specific binders may reduce the chance of false positives. Commercially available SEB specific antibodies or nonspecific resins may be used in combination with deliberately spiked SEB water samples. For example, fused silica capillaries coated with SEB antibody may effectively capture SEB (Koch S et al., (2000) Biosens Bioelectron 14, 779-784). The different immobilization options may bind to the different isoforms of the SEB toxin. Conditions sufficient or optimized for efficient phage-SEB binding, the removal of non-binding phage and the elution of phage specifically binding SEB may be provided.

Non-specific binding of the phage to the column and incomplete removal of non-specifically bound phage may result in false positives. Different column supports may be used to minimize non-specific phage binding. For example nitrocellulose membranes and impermeable glass or plastic beads and magnetic beads may be used. Solid supports may be housed in other constructs besides columns such as centrifugal devices, microplates, and capillary tubes to improve the efficiency of the assay. Because filamentous phage are resistant to extreme conditions, high stringency washes and elution solutions may be used. The wash may and elution solution may be selected to maximize phage elution without compromising phage infectivity or with acceptable compromise of phage infectivity. For example, phage are resistant to acidic buffers of pH 2.2, alkaline buffers such as 0.1 M triethylamine and urea concentrations up to 6 M. In addition, specific elution of the phage without releasing phage that may be bound non-specifically to the column may be achieved through competitive elution using excess purified peptide or other molecule which may compete with phage-SEB binding.

M13 phage specifically infects E. coli cells containing the F conjugative plasmid. The optimal conditions for generating phage susceptible F⁺ E. coli cells may be the mid stages of exponential growth. Active metabolism also may be desirable or required for luxAB expression and light emission. How quickly the reporter M13 phage will produce a detectable light signal upon infection may vary. However, the expression of M13 genes may be detected 2 minutes after phage infection and the release of phage progeny occurs 15-20 minutes later. In addition, listeria phage containing the luxAB reporter genes may produce a detectable signal 15-20 min after infection of listeria cells. Therefore, in certain embodiments, the time required to produce a signal after E. coli infection may be measured in minutes rather than hours. The amount of toxin present in the sample may be correlated to the amount and speed of signal produced.

In other embodiments, the E. coli may be genetically engineered to be rendered more susceptible to phage infection leading to faster response times. This may be achieved for example, by genetically engineering the E. coli to express more receptors (F pili) for the M13 phage (Malmborg et al., 1997. J. Mol. Biol. 273: 544-551). For example, E. coli only express up to 3 pili/bacterium under ideal conditions. Genetically modified bacteria that express more pili/bacterium may be more susceptible to phage infection. Since bacteria differentially express F pili at different stages of growth, continuous expression of F pili from a continuously expressed promoter in genetically engineered bacteria may also render them more injectable than wild type bacteria and may thus be more suitable for use in a kit. In addition, the sequence of the F pili may be deliberately mutated by error prone PCR for example, in order to obtain pili that are more resistant to environmental extremes. This may be useful for kit testing in a non laboratory environment.

In other embodiments, a peptide, antibody, nucleic acid, or fragments thereof that bind to any target of interest (e.g., toxin) may be placed on the phage coat. A detection system and method may be used for identification and detection of any biological molecule or entity, such as a virus, bacteria, protein, biomarker, or bioregulator. For example, peptides may be genetically engineered to specifically bind to inorganic compounds and may be used for the identification and detection of metals, oxides and other technological compounds.

Because phage display may screen billions of different peptides and identify high affinity peptide binders to the target of choice, phage display may be used to adapt the detection system and method to detect any toxin or biomarker of disease. Changes may also be made by changing the binder and column capture characteristics.

Peptides may also be designed to bind to multiple targets, particularly if the targets are similar, or the phage may be engineered to express more than one type of peptide. Alternatively, recombinant phages displaying unique peptides may be pooled to contain a mixed phage population, each potentially binding a different target molecule; the mixed recombinant phage each displaying unique peptides may also contain different reporter genes that produce different signals depending on the target molecule. This may allow simultaneous detection of more than one target molecule. Such detection may be particularly useful where the benefit of knowing quickly and with one test that any of a number of targets are present outweighs any determination of the specific target identity and/or amount.

The genetically engineered phage may be modified by changing the specificity of the displayed peptide on the phage coat using phage display methods. Additionally, the phage may be engineered to contain other reporter genetic material such as a fluorescent protein, including green fluorescent proteins, and the phage may be further engineered to enhance its utility as a detection tool. For example, the phage may be engineered to express a higher number of binding peptides on its coat.

The detection system and method may also be used in concert with automated aerosol collection techniques (e.g., Hindson B J et al., (2005) Anal Chem. 77, 284-289) for the detection of aerosolized SEB or other target molecules.

In a particular embodiment, a detection system and method may be used to detect bioregulatory molecules. Such detection may be important because these molecules may have the potential to function at very low doses and thus may be more difficult to detect in a useful manner.

In another embodiment, the detection system and method may be adapted to detect staphylococcus. Staphylococcal enterotoxin food poisoning may be one of the most common forms of food borne illness following the ingestion of contaminated food such as dairy products.

A system may include a column having an inner and outer surface, wherein the inner surface is at least partially coated with a binder molecule. A column may be in liquid communication with a loading chamber configured to apply solutions (e.g., test sample, wash solutions) to the column. A column may be in liquid communication with a collection chamber configured to receive liquid passed or forced through the column. A collection chamber may be configured to receive liquid from the column in one or more fractions.

### EXAMPLES

Some specific embodiments of the disclosure may be understood, by referring, at least in part, to the following examples. These examples are not intended to represent all aspects of the disclosure in its entirety. Variations will be apparent to one skilled in the art.

### Example 1

### Construction of the Recombinant luxABM13 Phage

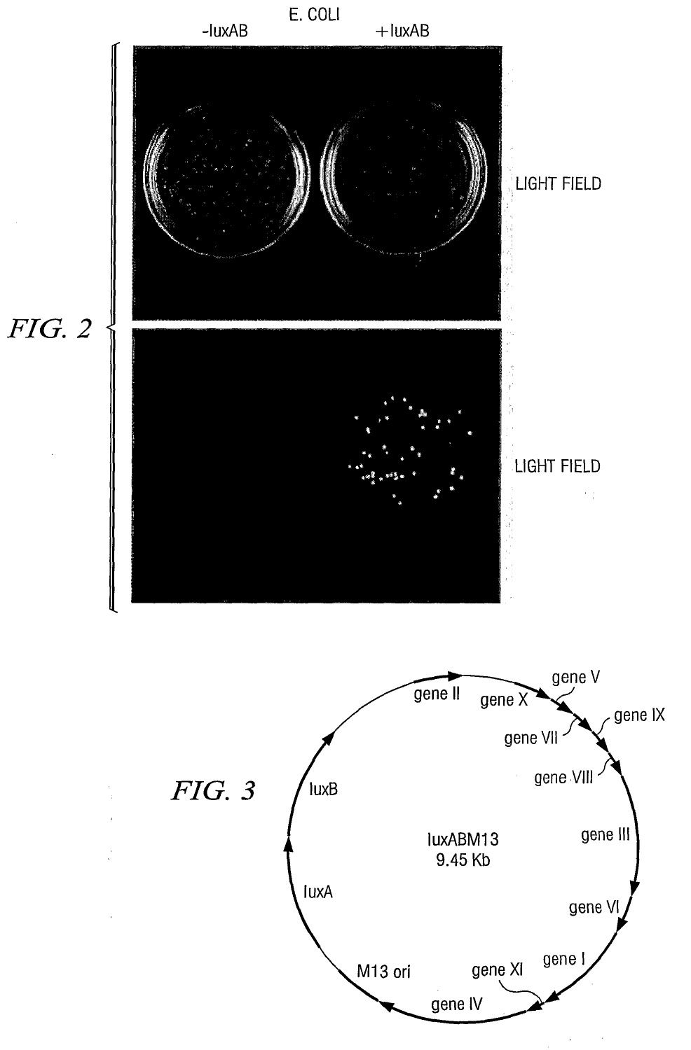

The Vibrio harveyi ‘light’ genes, luxA and luxB were PCR-amplified using pQF110 (ATCC77113) as template. The PCR primers were designed to contain Xba1/BamHI and HindIII/XhoI, respectively, for directional cloning into the corresponding sites of pSK−. The 5′ primers were also designed to contain a consensus E. coli ribosome binding site (TAAGGAGGTAAAAAA(ATG); SEQ ID NO. 2) (Schofield D A et al., (2002) FEMS Microbiol Lett 215, 237-42. An E. coli promoter containing consensus transcriptional signals (Schofield D A et al., (2003) Appl Environ Microbiol 69, 3385-3392) was cloned upstream (SacI/NotI sites) of luxAB and the transcriptional terminator TL17 (Wright et al. (1992) EMBO J. 11, 1957-64) was cloned downstream (Kpn I sites) of the luxA and luxB genes. To test whether the luxAB expression vector was functional, the luxAB plasmid was transformed into E. coli ER2738 and the resulting colonies were examined under dark field illumination in the presence of the substrate decanal. In the absence of luxAB, luminescent colonies were not obtained (FIG. 2); however, when luxAB was present, luminescent colonies were clearly visible. This demonstrated that the optimized luxAB cassette was functioning in E. coli. The luxAB expression cassette, which was flanked by Sph I sites, was then cloned into the same sites of bacteriophage vector M13KE (New England Biolabs) to create luxABM13 (FIG. 3).

### Example 2

### Analysis of the Efficiency of the Recombinant Phage

Cloning of the luxAB cassette into M13KE increased the genome size from approximately 7.2 kb to 9.4 kb. Although, fairly large fragments may be cloned into M13 without any loss of phage function, the size of DNA fragment, the orientation, and the sequence may negatively impact the ‘fitness’ of the phage. To check the ‘fitness’ of the recombinant phage, the burst size and the latency of the recombinant luxABM13 phage was compared to the wild-type M13 phage.

### Example 3

### Burst Size

The burst size may be the number of progeny phage released after the initial infection. E. coli ER2738 cultures (OD₆₀₀ of 0.605, 5.8×10⁸ CFU/mL) were infected with wild-type or recombinant phage at time 0 with a multiplicity of infection (MOI) of approximately 10. After 40 min, the culture supernatants were passed through a 0.2 μm filter and enumerated for phage using the soft agar overlay technique (Westwater et al. (2003) Antimicrob Agents Chemother 47, 1301-7).

The results indicated that the recombinant luxABM13 had a slightly lower burst size than the wild-type M13 phage (Table 1).

TABLE 1
Burst size of the wild-type M13 phage
and recombinant luxABM13 phage.

PFU/mL *(SD)

| Time (min) | M13 | luxABM13 |
|---|---|---|
| 0 | 7.5 × 10⁹(7.5 × 10⁷) | 7.6 × 10⁹(8.7 × 10⁷) |
| 40 | 1.6 × 10¹¹(2.0 × 10⁹) | 5.9 × 10¹⁰(1.5 × 10⁹) |

*Average of triplicate infections followed by the standard deviation in parentheses.

### Example 4

### Latency

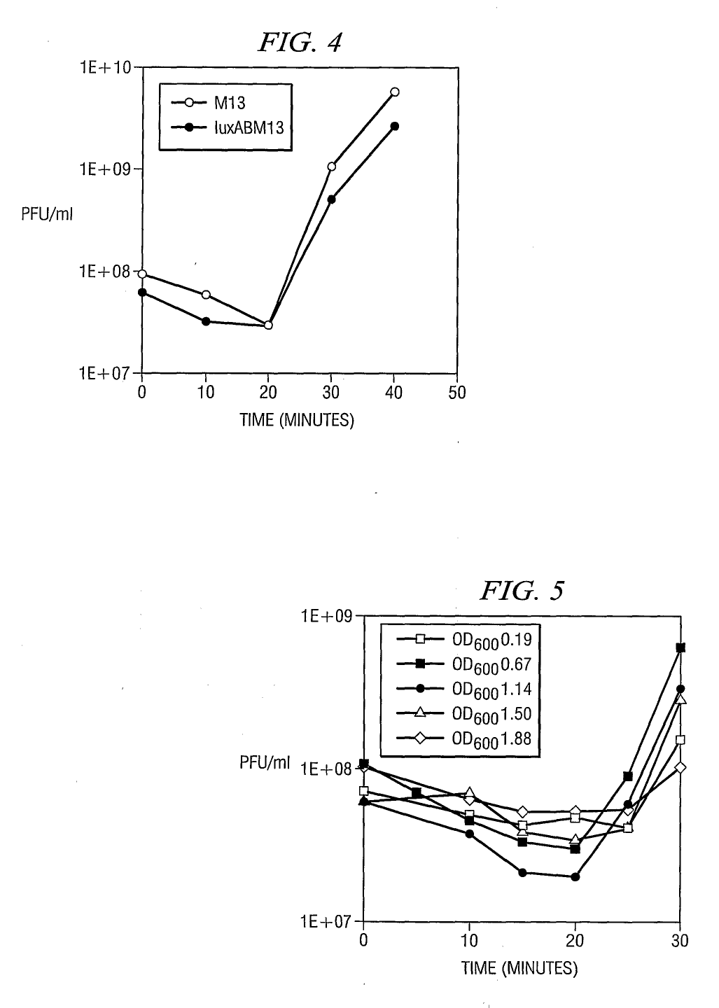

Latency may be the time from infection until the first new, progeny phage are released from the host cells. E. coli ER2738 cultures (OD₆₀₀ 0.645, 7.5×10⁸ CFU/ml) were infected at time 0 with an MOI of 0.124 and 0.084 for the wild-type and luxABM13 phages, respectively, and phage progeny release was monitored using the soft agar overlay technique. The results indicated that the latency period of luxABM13 was very similar to the wild-type M13 phage and occurred about 20 min after infection (FIG. 4).

### Example 5

### Optimal Stages of E. coli Growth for luxABM13 Infection

M13 phage specifically infects E. coli cells containing the F conjugative plasmid. The optimal conditions for generating phage susceptible F⁺ E. coli cells may be the mid stages of exponential growth. To verify that the mid stages of exponential growth were optimal for luxABM13 infection, E. coli ER2738 was harvested at various stages of growth, normalized to an OD₆₀₀ of approximately 0.7, and infected with luxABM13 (MOI of approx. 0.1). In agreement with the published literature for wild-type M13 phage, the mid stages (OD₆₀₀ 0.67-1.14) of E. coli exponential growth was also found to result in the shortest latency period for luxABM13 (FIG. 5).

### Example 6

### Optimal E. coli Concentrations for Phage Infection

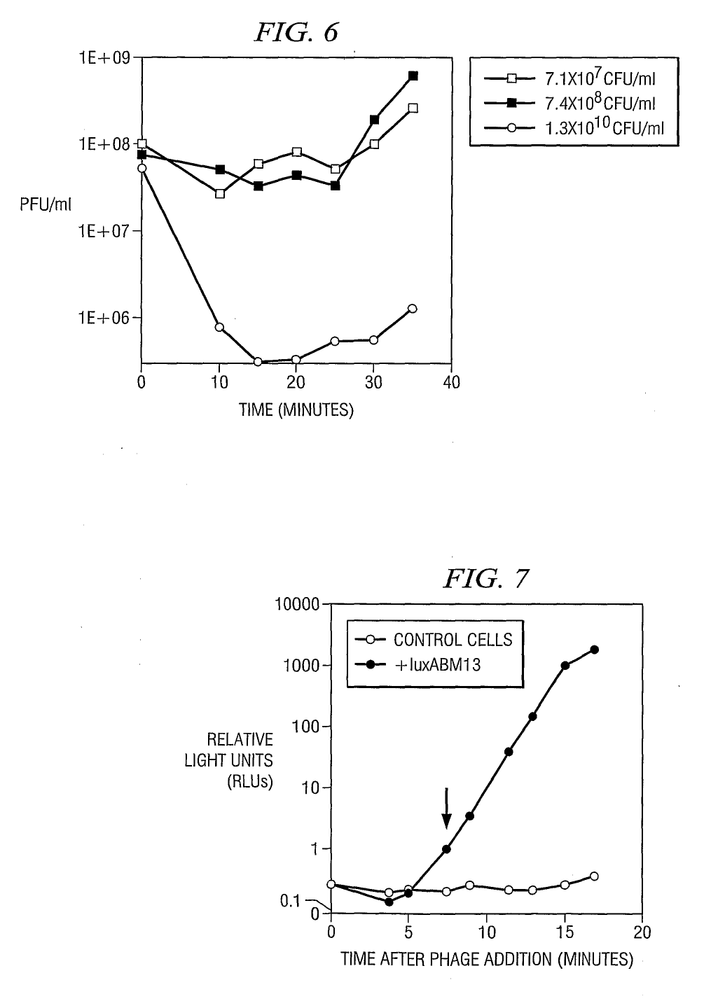

The density of the E. coli culture may be an important parameter affecting the speed with which a phage finds and infects a host. To determine a desirable concentration(s) for the phage/E. coli interaction, E. coli ER2738 was grown to an OD₆₀₀ of 0.65. The culture was harvested by centrifugation, and resuspended in prewarmed media to 0.1 of the original volume. The culture was then diluted as appropriate to generate a 10, 1, and 0.1 concentration of the original culture. Cultures (100 μl) were infected with 1×10⁷ luxABM13. The results indicated that a high density of E. coli (1×10¹⁰ CFU/ml), resulted in a quick decrease in the number of ‘available’ phage in the culture supernatant. This suggests that the high density was preferential for the phage/host interaction (FIG. 6).

### Example 7

### Time to Light Signal

M13 gene expression may be detected 2 minutes after phage infection while the release of phage progeny may occur 20 minutes later. listeria phage containing the luxAB reporter genes may produce a detectable light signal 15-20 min after infection of listeria cells. To investigate how quickly the luxABM13 phage may infect E. coli and produce a light signal, E. coli ER2738 cells were harvested at OD₆₀₀ 0.6, concentrated 10-fold (7.1×10⁹ CFU/ml), and infected with luxABM13 at an MOI of approximately 8. Cultures were harvested at the designated times after infection and monitored for light production using the luminometer LMII³⁸⁴. The data indicated that the signal response time (time period from phage infection to detectable light production above uninfected E. coli) was 7.5 min (FIG. 7).

### Example 8

### Relationship Between Amount of Input Phage and Light Output

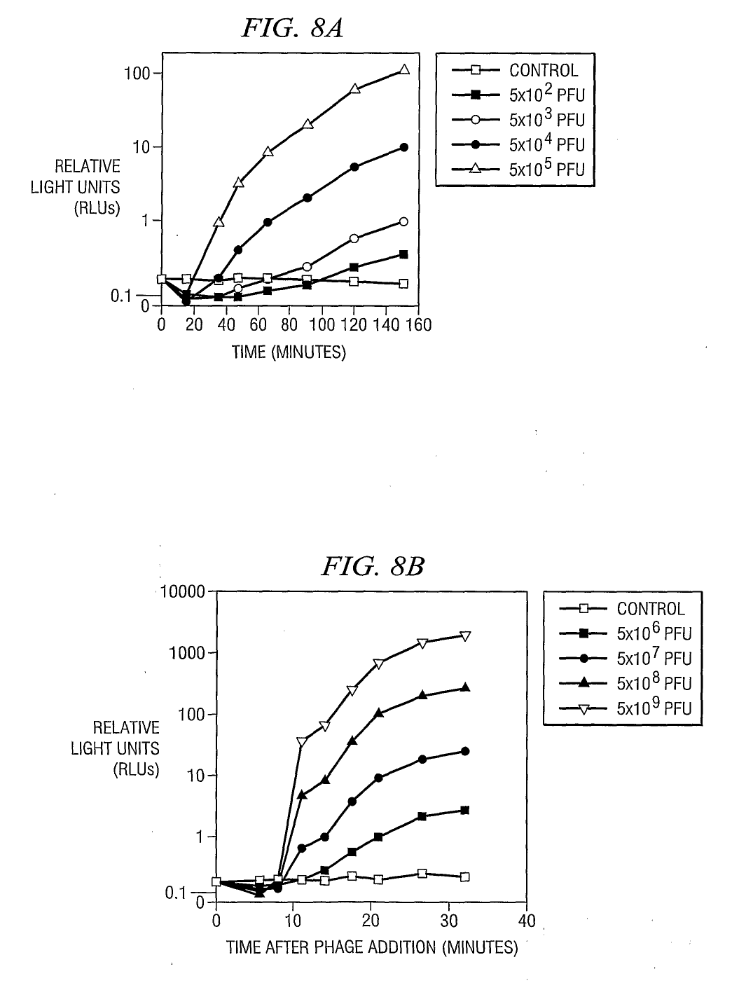

It may be important to be able to detect and also quantify (relatively) the target in the original sample. To investigate if the amount of light produced correlates with the amount of input phage, 10-fold serial dilutions were performed on the stock phage (10⁹-10² plaque forming units, (pfu/mL)) which was then used to infect E. coli ER2738 (OD₆₀₀ 0.6-0.7). As the amount of input phage decreased, the amount of light produced decreased (FIGS. 8A and 8B). In addition, as the amount of input phage decreased, the signal response time increased. This indicates that it should be possible to quantify (relatively) the amount of target in the original sample by the amount of light produced and the time taken to produce a light signal.

### Example 9

### Sensitivity

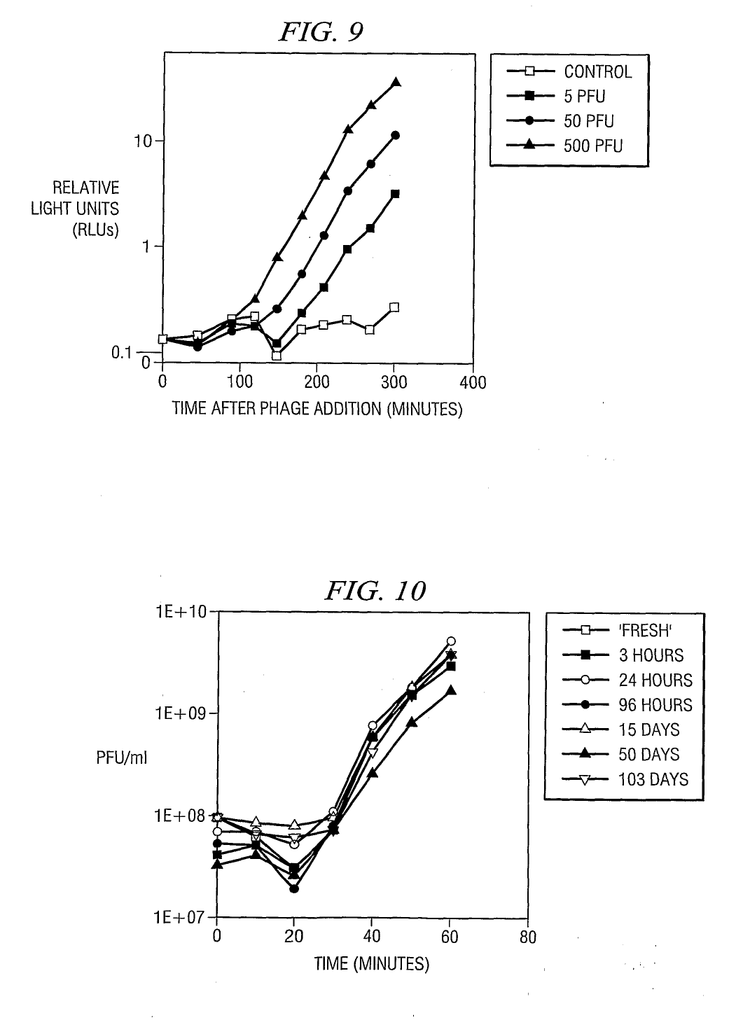

According to some embodiments, it may be desirable, important, and/or vital for the assay to be sensitive since the target may be present at very low concentrations. To determine the sensitivity limits of the assay, E. coli ER2738 (OD₆₀₀ of 0.35) were infected with 5, 50, and 500 luxABM13 pfu and monitored for light production over time (up to 300 min). As the number of input phage dropped from 500 to 5 pfu, the signal response time increased and the amount of light decreased (FIG. 9). Nevertheless, as few as 5 pfu may be detected, albeit after a longer signal response time (200 min). This indicates that the methodology has the potential (assuming efficient column binding and recovery) to detect single copies of the target molecule and in that respect, has sensitivity characteristics that are comparable to PCR.

### Example 10

### Effects of Freezing and Storage on Infectibility and Latency

Phages are resistant to environmental extremes and may be stored for months or years without a significant loss in phage infectivity. If phage susceptibility is lost after long-term storage, it may return although it is not clear in every case how long this may take. This may be an important consideration if the detection device is to be used outside of the laboratory and exhibit a quick response time. Therefore, experiments were performed that examined luxABM13 latency (time from infection to new phage progeny produced) using E. coli cells that have been stored frozen for varying lengths of time at −70° C. E. coli E 738 were grown overnight in Luria Bertani (LB) broth containing tetracycline (25 g/ml), diluted 1:100 (0.5 into 50 mL) and grown at 37° C. at 235 rpm. After 3-4 hours, cultures (OD₆₀₀ 0.650, 45 ml) were chilled on ice, harvested by centrifugation, resuspended in cold 1.5 ml 10% glycerol and flash frozen (100 μL aliquots) in dry ice/ethanol. Cells were stored at −70° C. until the time of experiment. At the designated storage times, cells (300 μl) were defrosted on ice, and mixed with prewarmed LB (9.7 mL). Cells were immediately measured (colony counting after overnight growth) for CFU (approx. 6×10⁸ CFU/ml) and infected with the luxABM13 phage at an MOI of approximately 0.1. At the designated times, culture supernatants (ice, 2 min bench top centrifuge, 0.2 μm filter) were enumerated for phage using the plaque assay (FIG. 10). LuxABM13 latency was compared using ‘fresh’ cells and cells which had been stored at −70° C. for up to 103 days. The results indicated that the luxABM13 latency period was very similar using either fresh or stored E. coli cells, irrespective of the length of storage time. Therefore, freezing and storage of E. coli ER2738 under the conditions tested does not significantly affect the time it takes for luxABM13 to infect and produce progeny phage.

### Example 11

### Light Production and Signal Response Time Using Stored E. coli Cells

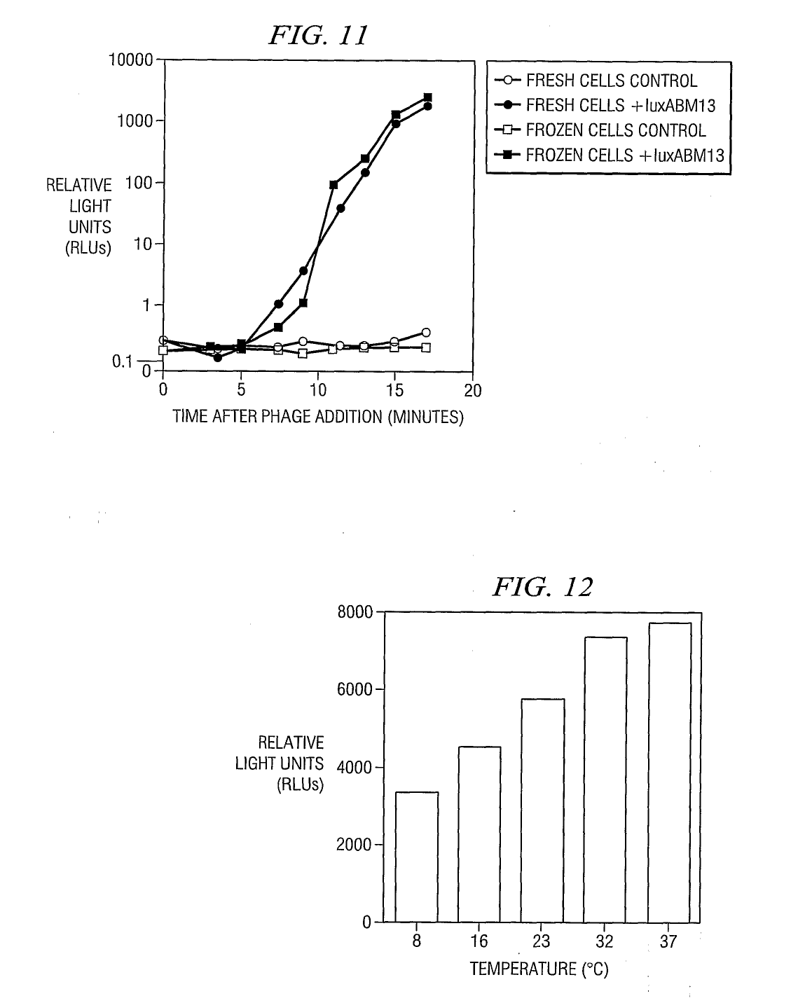

Active metabolism may be required for luxAB expression and light emission. Although the luxABM13 latency may be similar irrespective of whether fresh or frozen E. coli cells are used, it was of interest to examine if stored (frozen) cells negatively affected the ability of luxABM13 to produce light. Therefore, the signal response time of fresh and stored cells, infected with the luxABM13 phage, was examined. Freshly prepared (OD₆₀₀ of 0.6) or stored (frozen at −70° C. for 90 days) phage competent E. coli ER2738 cells, were infected with a MOI of 8 and 16, respectively and monitored for light production. The amount of light produced and the signal response time was similar irrespective of whether fresh or stored cells were used (FIG. 11). This indicates that prior storage at −70° C. for 90 days does not negatively affect the ability of E. coli ER2738 to be infected with luxABM13 and produce light.

### Example 12

### Temperature Sensitivity of E. coli Produced luxAB Proteins

In Listeria monocytogenes LuxAB protein synthesis and stability may be temperature sensitive. Maximum light production occurred at 20° C., while incubations at higher temperatures (30 and 37° C.) significantly reduced luminescence. To investigate whether light production was temperature sensitive in E. coli, E. coli ER2738 harboring a pBLUESCRIPT plasmid containing the luxAB cassette, was grown until mid log phase at 32° C. Equal cell aliquots were then incubated at the designated temperatures for 10 min prior to assaying for luciferase (light) production. The results indicated that light production in E. coli was temperature dependent (FIG. 12); however, in contrast to Listeria, maximum light production was observed at elevated (37° C.) temperatures, and activity decreased as the temperature decreased. These results are for a short (10 min) incubation at the designated temperatures, and are independent of infection, but are dependent on E. coli growth, and gene/protein expression. Repeat experiments should be performed examining the activity of enzyme lysates at the different temperatures.

While embodiments of this disclosure have been depicted, described, and defined by reference to specific example embodiments of the disclosure, such references do not imply a limitation on the disclosure, and no such limitation is to be inferred. The subject matter disclosed may be capable of considerable modification, alteration, and equivalents in form and function, as will occur to those ordinarily skilled in the pertinent art and having the benefit of this disclosure. For example, a variety of phage and bacteria pairs may be used. Other viruses and non-bacterial cells (e.g., yeast) may even be used in some embodiments. Similarly, a number of different types of reporter genetic material and detection systems may be used. The depicted and described embodiments of this disclosure are examples only, and are not exhaustive of the scope of the disclosure.

As will be understood by those skilled in the art, other equivalent or alternative methods, devices, systems and compositions for detecting the presence of a target molecule according to embodiments of the present disclosure may be envisioned without departing from the essential characteristics thereof. For example, where a range is disclosed, the end points may be regarded as guides rather than strict limits. In some embodiments, methods, compositions, devices, and/or systems may be adapted to accommodate ergonomic interests, aesthetic interests, scale, or any other interests. Such modifications may influence other steps, structures and/or functions (e.g., positively, negatively, or insubstantially). A negative influence on function may include, for example, a loss of fractionation capacity and/or resolution. Yet, this loss may be deemed acceptable, for example, in view of offsetting ergonomic, aesthetic, scale, cost, or other factors.

In some embodiments, a device of the disclosure may be manufactured in either a handheld or a tabletop configuration, and may be operated sporadically, intermittently, and/or continuously. Individuals skilled in the art would recognize that additional separation methods may be incorporated, e.g., to partially or completely remove proteins, lipids, carbohydrates, nucleic acids, salts, solvents, detergents, and/or other materials from a test sample. Also, the temperature, pressure, and acceleration (e.g., spin columns) at which each step is performed may be varied.

All or part of a system of the disclosure may be configured to be disposable and/or reusable. From time to time, it may be desirable to clean, repair, and/or refurbish at least a portion of a device and/or system of the disclosure. For example, a reusable component may be cleaned to inactivate, remove, and/or destroy one or more contaminants. Individuals skilled in the art would recognize that a cleaned, repaired, and/or refurbished component is within the scope of the disclosure. In addition, individuals skilled in the art would recognize that a fractionator may further comprise an elution detector (e.g., an optical, spectrophotometric, fluorescence, and/or radioisotope detector) configured to monitor removal (e.g., elution) of phage from the binder.

These equivalents and alternatives along with obvious changes and modifications are intended to be included within the scope of the present disclosure. Moreover, one of ordinary skill in the art will appreciate that no embodiment, use, and/or advantage is intended to universally control or exclude other embodiments, uses, and/or advantages. Expressions of certainty (e.g., “will,” “are,” and “can not”) may refer to one or a few example embodiments without necessarily referring to all embodiments of the disclosure. Accordingly, the foregoing disclosure is intended to be illustrative, but not limiting, of the scope of the disclosure.

1. A detection system comprising:
a phage having a surface molecule with a binding affinity for a target molecule;
a binder with a binding affinity for a target molecule;
a phage wash composition operable to separate unbound phage from the binder;
a release composition operable to separate phage from the binder;
a bacterium operable to take up at least a portion of the phage; and
a detector configured to differentiate between cells that have taken up at least a portion of a phage and cells that have not.

2. A system according to claim 1, wherein the target molecule comprises a toxin.

3. A system according to claim 2, wherein the target molecule comprises Staphylococcal enterotoxin B.

4. A system according to claim 1, wherein the target molecule comprises an amino acid.

5. A system according to claim 1, wherein the target molecule comprises a nucleic acid.

6. A system according to claim 1, wherein the target molecule comprises a lipid.

7. A system according to claim 1, wherein the target molecule comprises a carbohydrate.

8. A system according to claim 1, wherein the target molecule comprises a metal.

9. A system according to claim 1, wherein the dissociation constant of the binder for the target molecule is over about 10⁻⁶ M.

10. A system according to claim 1, wherein the binder comprises an antibody.

11. A system according to claim 1, wherein in the binder comprises a column.

12. A system according to claim 1, wherein the phage comprises a reporter nucleic acid and the detector is configured to detect at least a portion of the reporter nucleic acid.

13. A system according to claim 1, wherein the reporter nucleic acid comprises luxAB reporter genes.

14. A system according to claim 1, wherein the phage comprises a non-lytic phage.

15. A system according to claim 14, wherein the phage comprises M13 and the bacterium comprises Escherichia coli.

16. A system according to claim 14, wherein the Escherichia coli is susceptible to phage infection.

17. A system according to claim 1, wherein the phage comprises a phagemid vector.

18. A system according to claim 1, wherein the phage comprises a lytic phage.

19. A system according to claim 1, wherein the bacterium comprises reporter nucleic acid.

20. A system according to claim 19, wherein the reporter nucleic acid is operably linked to an expression control sequence, and wherein the phage comprises a nucleic acid encoding a protein operable to positively or negatively effect the expression control sequence.

21. A system according to claim 19, wherein the expression control sequence comprises a promoter that is active only in the presence of an activator protein, and wherein the phage comprises an expressible nucleic acid encoding the activator protein.

22. A method of detecting the presence of a target molecule in a test sample comprising:
contacting a binder with the test sample to form a binder—test sample mixture under conditions that permit binder—target molecule binding;
contacting the binder—test sample mixture with a phage having a reporter nucleic acid, wherein the phage is capable of binding the target molecule; under conditions that permit phage—target molecule binding to form a binder—sample—phage mixture,
washing the binder—sample—phage mixture under conditions that remove phage not bound to the target molecule from the binder (e.g., using a phage wash composition) to form a washed binder—sample—phage mixture;
treating the washed binder—sample—phage mixture to release phage bound to the target molecule, if any, from the binder to form a released phage composition;
contacting a bacterium with the released phage composition under conditions that permit uptake of at least a portion of the released phage, if present, by the bacterium, and
detecting the presence in the bacterium of the reporter nucleic acid.
wherein the presence of reporter nucleic acid in the bacterium is indicative of presence of the target molecule in the test sample, while absence of reporter nucleic acid in the bacterium is indicative of absence of the target molecule in the test sample.

23. A method according to claim 22, wherein the target molecule comprises a toxin.

24. A method according to claim 22, wherein the target molecule comprises Staphylococcal enterotoxin B.

25. A method according to claim 22, wherein the binder comprises a binder specific for the target molecule.

26. A method according to claim 22, wherein the binder comprises an antibody.

27. A method according to claim 22, wherein in the binder comprises a column.

28. A method according to claim 22, wherein the reporter genetic material comprises the luxAB reporter genes.

29. A method according to claim 22, wherein detecting the presence of any reporter nucleic acid in the bacterium comprises detecting luminescence.

30. A method according to claim 22, further comprising detecting an amount of any reporter nucleic acid in the bacterium, wherein the amount of reporter nucleic acid in the bacterium is indicative of an amount of the target molecule in the test sample.

31. A method according to claim 22, wherein the phage comprises a non-lytic phage.

32. A method according to claim 22, wherein the phage comprises M13 and the bacterium comprises Escherichia coli.

33. A method according to claim 22, wherein the phage comprises a phagemid vector.

34. A method according to claim 33, further comprising assembling the phage by packaging the phagemid vectors into a phage with the aid of a helper phage.

35. A method of detecting the presence of a target molecule comprising:
placing a sample suspected of containing the target molecule in conditions operable to allow binding of the target molecule to a binder;
placing the binder in the presence of a genetically engineered phage operable to bind the target molecule;
washing phage not bound to the target molecule from the binder;
releasing phage bound to the target molecule from the binder;
infecting a bacterium having reporter genetic material with the released phage; and
detecting the a change in expression of the reporter genetic material in the bacterium;
wherein a change in expression of reporter genetic material in the bacterium is indicative of presence of the target molecule in the sample, while absence of a change in expression of reporter genetic material in the bacterium is indicative of absence of the target molecule in the sample.

36. A method according to claim 35, wherein the reporter genetic material is under control of a promoter active only in the presence of an activator protein, and wherein the phage has genetic material encoding the activator protein.

37. A method according to claim 35, wherein the reporter genetic material is under the control of a repressible promoter, and wherein the phage has genetic material encoding a protein operable to repress or unrepress the promoter.

38. A method of detecting the presence of a target molecule comprising:
placing a sample suspected of containing the target molecule in conditions operable to allow binding of the target molecule to a binder;
placing the binder in the presence of a genetically engineered phage operable to bind the target molecule;
washing phage not bound to the target molecule from the binder;
releasing phage bound to the target molecule from the binder;
infecting a bacterium with the released phage; and
detecting infection of the bacterium;
wherein infection of the bacterium is indicative of presence of the target molecule in the sample, while absence of infection of the bacterium is indicative of absence of the target molecule in the sample.

39. A method according to claim 38, wherein the phage comprises a lytic phage.

40. A method according to claim 39, wherein detecting infection of the bacterium comprises detecting lysis of the bacterium.

41. A method according to claim 38, wherein the phage comprises a non-lytic phage.

42. A method according to claim 38, wherein detecting infection of the bacterium comprises detecting progeny phage.

43. A kit comprising:
a genetically engineered phage, wherein the phage expresses a surface molecule operable to bind a target molecule;
a binder operable to bind the target molecule; and
a bacterium susceptible to infection by the phage;
wherein infection of a bacterium by a phage is indicative of phage binding to the target molecule.

44. A kit according to claim 43 further comprising a detection component operable to determine whether the bacterium has been infected by the phage.

45. A kit according to claim 43 further comprising an a bacterium engineered to be resistant to environmental extremes.
